(19) United States
(12) Patent Application Publication
(10) Pub. No.: US 2009/0275904 A1
(43) Pub. Date: Nov. 5, 2009

(54) SHEET ASSEMBLIES WITH RELEASABLE MEDICAMENTS

(76) Inventors: Neil Rajendra SARDESAI, Arcadia, CA (US); Matthew Thomas Borzage, Chico, CA (US)

Correspondence Address: LADAS & PARRY, 5670 WILSHIRE BOULEVARD, SUITE 2100, LOS ANGELES, CA, 90036-5679, US

(21) Appl. No.: 12/267,369

(22) Filed: Nov. 7, 2008

Related U.S. Application Data
(60) Provisional application No. 61/050,144, filed on May 2, 2008.

(51) Int. Cl. A61M 35/00; D06M 17/00; A41D 1/00

(52) U.S. Cl. 604/289; 156/313; 2/69

(57) ABSTRACT

A sheet assembly comprises an insoluble layer disposed to define an inner reservoir and a medicament disposed in the inner reservoir. A garment is formed from said textile assembly.

### RELATED APPLICATIONS

This patent application claims the priority of co-pending U.S. Provisional Patent Application Ser. No. 61/050,144, filed on May 2, 2008, the entire contents of which are incorporated herein in their entirety by reference thereto.

### TECHNICAL FIELD

This disclosure relates to wound treatment and to garments, and more particularly to garments that treat wounds suffered by the wearer of the garments.

### BACKGROUND

Soldiers, police officers, and others who put themselves regularly in harm's way wear a variety of protective gear intended to protect them from being wounded by projectiles, explosions, knives, and the like. Such equipment can be life saving but does not always provide full protection and rarely provides full coverage. Certain body areas, such as the groin area, are usually not protected by typical body armor. It would be desirable and useful to provide instant treatment to wounds suffered by an individual without any human intervention. It would be further desirable for such treatment to be provided by equipment carried by the individual at all times, with little or no extra effort or encumbrance. The embodiments of the present disclosure answer these and other needs.

### SUMMARY

In a first embodiment disclosed herein, a sheet assembly comprises an insoluble layer disposed to define an inner reservoir and a medicament disposed in the inner reservoir.

In another embodiment disclosed herein, a method of forming a sheet assembly comprises disposing an insoluble layer so as to define an inner reservoir and disposing a medicament in the inner reservoir.

In a further embodiment disclosed herein, a method for providing a garment comprises disposing an insoluble layer so as to define an inner reservoir, disposing a medicament in the inner reservoir, and forming a garment from the insoluble layer containing the medicament.

These and other features and advantages will become further apparent from the detailed description and accompanying figures that follow. In the figures and description, numerals indicate the various features, like numerals referring to like features throughout both the drawings and the description.

### BRIEF DESCRIPTION OF THE DRAWINGS

FIG. 1(a) is a side view, in section, of a sheet assembly in accordance with the present disclosure;

FIG. 1(b) is a top view of the sheet assembly of FIG. 1(a);

FIG. 2 is a side view of a garment worn by a user and formed with a sheet assembly in accordance with the present disclosure;

FIG. 3(a) is a side view, in section, of a sheet assembly in accordance with the present disclosure;

FIG. 3(b) is a top view of the sheet assembly of FIG. 3(a);

FIG. 4 is a side view, in section, of a sheet assembly in accordance with the present disclosure;

FIG. 5 is a side view, in section, of a sheet assembly in accordance with the present disclosure;

FIG. 6(a) is a side view of a garment worn by a user and formed with a sheet assembly in accordance with the present disclosure; and

FIG. 6(b) is a side view, in section, of a detail of the garment of FIG. 6(a).

### DETAILED DESCRIPTION

In the following description, numerous specific details are set forth to clearly describe various specific embodiments disclosed herein. One skilled in the art, however, will understand that the presently claimed invention may be practiced without all of the specific details discussed below. In other instances, well known features have not been described so as not to obscure the invention.

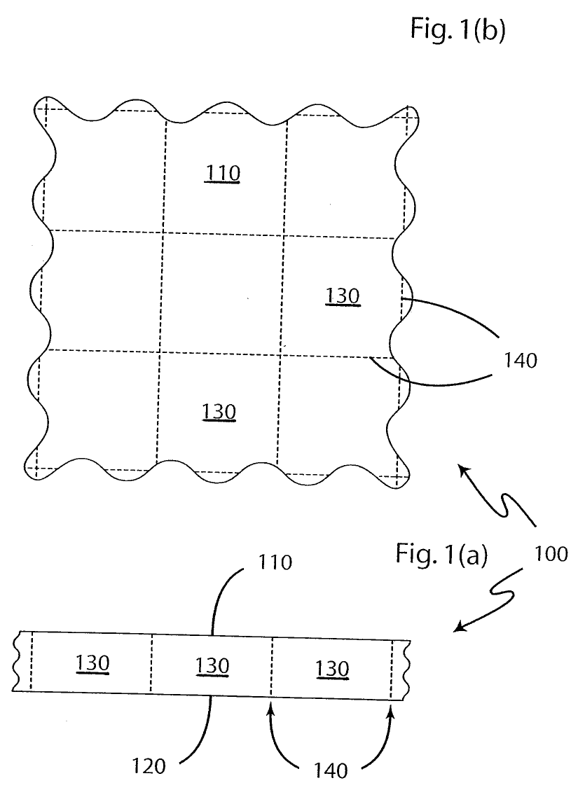

Referring to FIG. 1(a), a side view of a sheet assembly 100 in accordance with the present disclosure may be formed with a top 110 and a bottom 120 surface with a reservoir 130 defined therebetween. Optional apertures 140 may be provided between the top and bottom surfaces of the sheet assembly to allow moisture to flow therebetween, and/or to aid in tearing the textile assembly. In the top view of the embodiment of FIG. 1(b) of the sheet assembly 100, the apertures 140 extend across the surface of the assembly in a regular pattern. It must be understood that the paths along which these apertures are formed may be in any configuration and are in no way intended to be constrained by the illustrated embodiments.

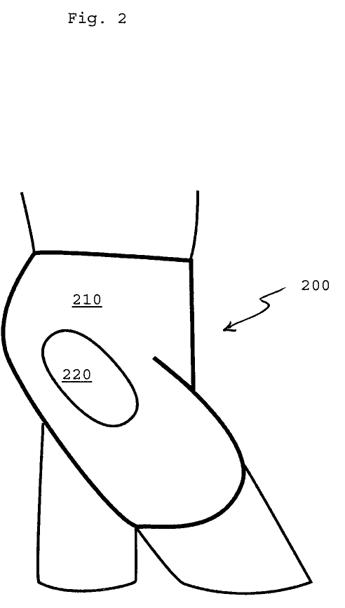

In a preferred embodiment, the reservoir 130 contains a quantity of a medicament therein that may be released upon puncture, tearing, melting or burning of the top and bottom surfaces of the assembly, such as may be caused by a projectile, sharp instrument, the force and/or heat of an explosion, or acids and other caustic substances. In other embodiments, the contents of the reservoir are at a higher pressure relative to the atmospheric pressure. As shown in FIG. 2, a preferred use of a sheet assembly 100 is in a garment such as an undergarment 200 that may be worn by a user (e.g. soldier) such that, upon the soldier being wounded in part of the body that is covered by the garment 200, medicament can seep from the reservoir 130 through the puncture in the sheet assembly 100 and into the wound sustained by the wearer/user of the garment. The garment 200 may be comprised entirely of the textiles assembly 100, or may be formed in any combination of the sheet assembly 100 and any other material. For purposes of illustration only, the garment 200 depicted in FIG. 2 is formed with two distinct portions 210 and 220. In one embodiment, only portion 220 may be formed with the sheet assembly 100. In other embodiments, both portions 210 and 220 may be formed with the sheet assembly 100 but to different degrees, e.g. portion 220 may contain a thicker layer of sheet assembly 100 or an embodiment of sheet assembly 100 that is formed with a thicker/more voluminous reservoir 130. In further embodiments other layers of textiles and other materials may be disposed over, below, or over and below the sheet assembly 100 in the formation of a garment 200. It must be understood that the specific application, use, or disposition of the sheet assembly 100 in a garment is in no way intended as a limitation upon the novel concepts disclosed herein.

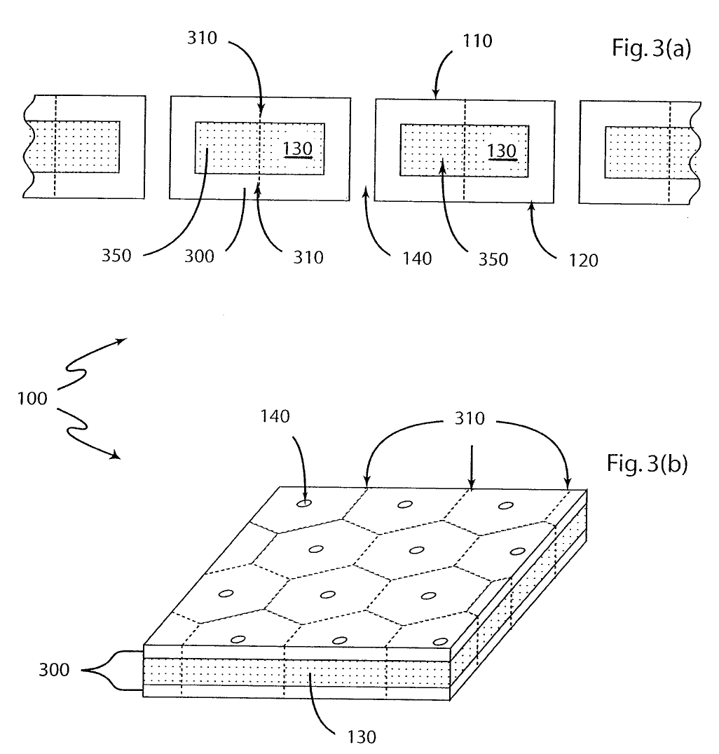

Referring to FIG. 3, an embodiment of sheet assembly 100 according to the present disclosure is shown in greater detail, and includes a layer 300 from which the top 110 and bottom 120 surfaces are formed. Optional apertures 140 may extend between the top and bottom surfaces and are likewise defined by the layer 300. The layer 300 is preferably impervious to the contents 350 contained in the reservoir 130 defined by the layer, such that the contents are not emitted from the reservoir during normal use of the sheet assembly such as wearing of a garment formed with the sheet assembly 100. The layer 300 is preferably insoluble, and may be formed of woven fabrics, extruded sheets, laminates, polymeric films, leathers, cellulose products, and any combination thereof. In one embodiment, the layer 300 is formed with elastic properties that allow it to stretch and thereby form a tight fit when worn by a user, thereby imparting a degree of pressure onto the inner reservoir that may aid in delivering the contents 350 of the reservoir to a wound site. In yet other embodiments, an elastic layer may be provided over the sheet assembly 100 to provide such additional pressure, such as an elastic waistband or the like.

In further embodiments in accordance with the present disclosure, additional optional apertures or slits 310 may be formed through or partially through the top and/or bottom surfaces to aid in tearing of the textile material. Such tearing apertures 310 would preferably be formed with sufficiently small dimensions to prevent the contents 350 of the reservoir 130 from leaching out. The pattern in which the apertures or slits 140 and 310 are formed is in no way a limitation upon the novel concepts disclosed herein. Slits 310 would preferably be formed only partially through the respective surface(s).

The actual disposition of the layer 300 in forming the inner reservoir 130 is also immaterial to the main novel concepts disclosed herein. The embodiments of FIGS. 1 and 3 are formed with two separate surfaces, each comprised of layer 300. In further embodiments, the top surface may be formed of a different material than the bottom surface; for example, the bottoms surface may be formed of a material that aids in wicking moisture away from the skin of the wearer, whereas the top surface may be formed of a material more impervious to moisture, sunlight, radiation, heat, dust, etc. In other embodiments, the top surface may be formed from a highly elastic material that evinces self-sealing properties to at least partially seal the tope of a puncture through the textile assembly, whereas the bottom surface may be formed from a substantially plastically deformable material that will maintain any puncture therethrough in an open state to allow the contents of the reservoir uninterrupted access to the wounded tissue of the user.

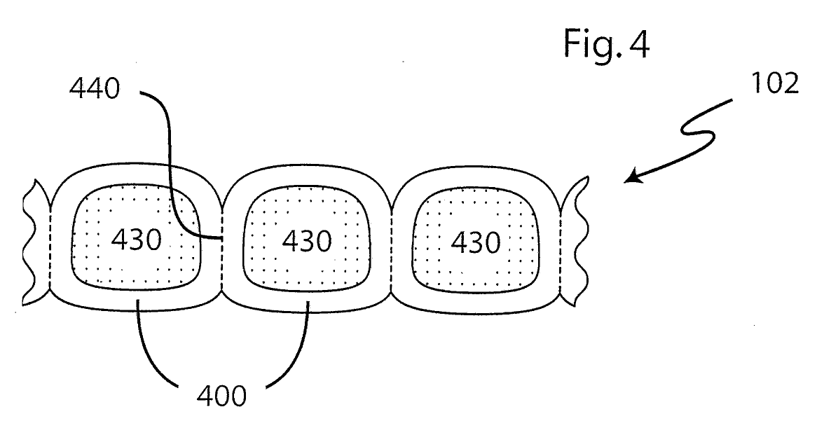

In still further embodiments—and with reference now to FIG. 4—a sheet assembly 102 according to the present disclosure may be formed with a layer 400 disposed to define all boundaries of a plurality of finite inner reservoirs 430. Such finite reservoirs may be formed with a relatively narrow cross section but with relatively long depth, or with any other combination of spatial dimensions as may best suit a garment to be formed of such a sheet assembly 102. As shown, such finite reservoirs 430 may be attached along interfaces 440 by any means practicable, including but not limited to gluing, taping, melding, sewing, welding such as ultrasonic welding, and any combination thereof, to thereby form sheet assembly 102. The attachment along interfaces 440 may be formed to offer limited resistance to tearing, thereby aiding the tearing of the sheet assembly by a user.

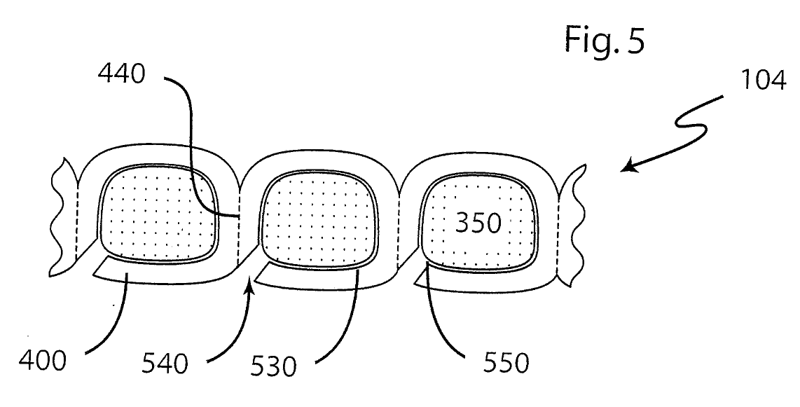

In a still further embodiment as illustrated in FIG. 5, a sheet assembly 104 may be formed as detailed previously with respect to sheet assembly 102 of FIG. 4, but may further include access ports 540 for access into each inner reservoir 530. In this embodiment, capsules or pouches 550 holding contents 350 may be inserted and removed through access port 540 to thereby allow the sheet assembly to be washed and otherwise handled without any premature, accidental release of contents 350. In this embodiment, capsules 550 are preferably formed from a material that is impervious to the contents 350, thereby minimizing or eliminating the need for the sheet assembly layers 400 to be impervious to the contents of their inner reservoirs 530. Although the embodiment illustrated depicts a capsule 550 that substantially fills the inner reservoir 530, any other size of capsules may be employed provided that they are large enough to be retained within the reservoir without being accidentally ejected through the access ports 540 during normal use of the textile assembly. It must be understood that the embodiments of FIGS. 4 and 5 may also be formed with larger apertures such as apertures 140 of the embodiment of FIG. 3 that are surrounded and defined by layer 400 or 500, to allow moisture to pass therethrough.

In a still further embodiment of a use of a sheet assembly in accordance with the present disclosure, a garment may be formed to incorporate such a sheet assembly and to further include pumping devices connected to pressurize the inner reservoir(s) of the sheet assembly and thereby aid in delivering the contents of the reservoirs to a wound site. Such pumping devices may be powered, such as by a battery or compressed gas, or manual, such as by the user's hand action. In one embodiment a garment such as a pair of pants may be formed with such pumping devices disposed in the buttocks area such that each time the user sits down, the pumps are actuated to deliver a quantity of pressurized air to the inner reservoir(s) of the sheet assembly incorporated into the pants, to thereby maintain the reservoir(s) under constant pressure.

As previously noted, the contents 150 of the inner reservoirs of the textile assemblies of the present disclosure preferably include one or more medicaments, which may be selected without limitation from among hemostatic agents, pain relief agents, antiviral agents, antibacterial agents, antibodies, antimicrobial agents, and any combination thereof. Such medicaments may take any physical form desirable, including without limitation liquids, gels, ointments, salves, pastes, powders, solids, and any combination thereof. The medicaments may be entrained in other materials such as preservatives, fixatives, surfactants, stabilizers.

In other embodiments, the inner reservoirs of the textile assemblies of the present disclosure may contain wound dressings and treatment preparations as currently known in the art. Examples of such wound dressings may be found, inter alia, in U.S. Pat. No. 7,351,422 to Jo et al., U.S. Pat. No. 7,303,759 to Mershon, U.S. Pat. No. 7,279,177 to Looney et al., U.S. Pat. No. 7,252,837 to Guo et al., U.S. Pat. No. 7,022,125 to Boethius, U.S. Pat. No. 6,762,336 to MacPhee et al., U.S. Pat. No. 5,645,849 to Pruss et al., U.S. Pat. No. 4,738,849 to Sawyer, and published U.S. Pat. App. Nos. 20080097271 to Lo et al., 20080020026 to Cochrum et al., 20070160653 to Fischer et al., 20050123588 to Zhu et al., 20040120993 to Zhang et al., and 20030149406 to Martineau et al., the entire contents of which are incorporated herein by reference thereto.

In a method of use of a sheet assembly according to the present disclosure, a garment may be formed to include the textile assembly. The garment may preferably be formed to be worn next to the user's skin, i.e. as an undergarment. A user may put such a garment on and, upon suffering a wound in an area covered by the garment from an attack that penetrates the garment, medicaments contained in the inner reservoir(s) of the sheet assembly incorporated into the garment will flow out of the reservoir and contact the open wound suffered by the user, thereby providing instant treatment to the user without any further intervention or assistance required from others. Optionally, the user may tear a portion of the sheet assembly from a non-impacted area of the garment to thereby release additional medicament, which the user can then manually apply to the wound. Optionally, the garment may be equipped with pumps to aid the delivery of the medicament to the wound; if such pumps are manually operated, the user or an assistant may actuate the pumps to thereby increase the flow of medicament to the wound site.

In a still further embodiment, a garment may be formed according to the present disclosure by incorporating a sheet assembly as described hereinabove in a portion of the garment, and extending conduits from the reservoir(s) in the sheet assembly portion to other portions of the garment to deliver their contents thereto upon suffering a penetrating injury. Such an embodiment may further incorporate means for pressurizing the reservoir(s) as described elsewhere herein.

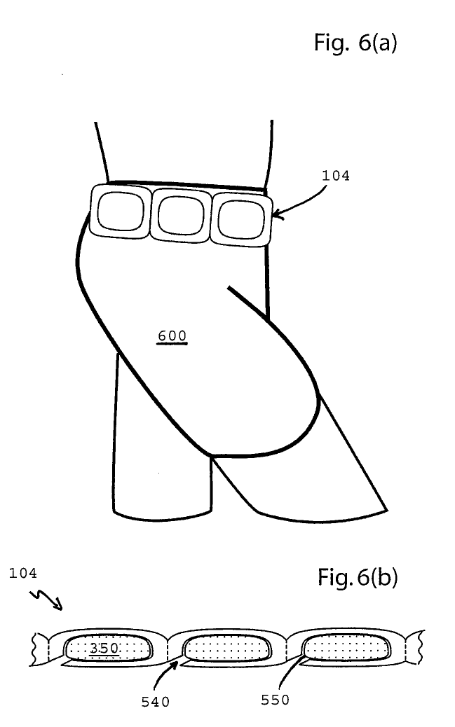

In a further embodiment illustrated in FIG. 6(a), a garment 600 may be formed of a sheet assembly 100, 102, 104 as described elsewhere herein, wherein the sheet assembly is disposed adjacent and surrounding the user's abdominal area when the user is wearing the garment. In one particular embodiment, the garment 600 is formed with a portion including a sheet assembly 104 which, as described herein above, includes access ports 540 for access into each inner reservoir 530. In this embodiment, capsules or pouches 550 holding contents 350 may be inserted and removed through access port 540 to thereby allow the garment to be washed and otherwise handled without any premature, accidental release of contents 350.

Having now described the invention in accordance with the requirements of the patent statutes, those skilled in this art will understand how to make changes and modifications to the present invention to meet their specific requirements or conditions. Such changes and modifications may be made without departing from the scope and spirit of the invention as disclosed herein.

The foregoing Detailed Description of exemplary and preferred embodiments is presented for purposes of illustration and disclosure in accordance with the requirements of the law. It is not intended to be exhaustive nor to limit the invention to the precise form(s) described, but only to enable others skilled in the art to understand how the invention may be suited for a particular use or implementation. The possibility of modifications and variations will be apparent to practitioners skilled in the art. No limitation is intended by the description of exemplary embodiments which may have included tolerances, feature dimensions, specific operating conditions, engineering specifications, or the like, and which may vary between implementations or with changes to the state of the art, and no limitation should be implied therefrom. Applicant has made this disclosure with respect to the current state of the art, but also contemplates advancements and that adaptations in the future may take into consideration of those advancements, namely in accordance with the then current state of the art. It is intended that the scope of the invention be defined by the Claims as written and equivalents as applicable. Reference to a claim element in the singular is not intended to mean “one and only one” unless explicitly so stated. Moreover, no element, component, nor method or process step in this disclosure is intended to be dedicated to the public regardless of whether the element, component, or step is explicitly recited in the Claims. No claim element herein is to be construed under the provisions of 35 U.S.C. Sec. 112, sixth paragraph, unless the element is expressly recited using the phrase “means for . . . ” and no method or process step herein is to be construed under those provisions unless the step, or steps, are expressly recited using the phrase “comprising the step(s) of . . . .”

What is claimed is:

1. A sheet assembly, comprising:
an insoluble first layer disposed to define an inner reservoir; and
a medicament disposed in the inner reservoir.

2. The sheet assembly of claim 1, further comprising:
an insoluble second layer disposed to cooperate with the first layer to define the inner reservoir therebetween.

3. The sheet assembly of claim 1, wherein the first layer comprises:
an insoluble first layer formed with a plurality of apertures spaced from the inner reservoir.

4. The sheet assembly of claim 1, wherein the first layer comprises:
an insoluble first layer formed with a plurality of apertures spaced from the inner reservoir and linearly aligned along at least one predefined path.

5. The sheet assembly of claim 1, wherein the first layer comprises:
an insoluble first layer disposed to define a plurality of inner reservoirs and formed with a plurality of apertures between the inner reservoirs; and
a medicament disposed in each inner reservoir.

6. The sheet assembly of claim 1, wherein the first layer comprises:
an insoluble first layer disposed to define a plurality of inner reservoirs and formed with a plurality of apertures aligned along at least one predefined path extending between the inner reservoirs; and
a medicament disposed in each inner reservoir.

7. The sheet assembly of claim 1, wherein the medicament is selected from the group consisting of hemostatic agents, pain relief agents, antiviral agents, antibacterial agents, antibodies, antimicrobial agents, and any combination thereof.

8. The sheet assembly of claim 7, wherein the medicament is selected from the group consisting of liquids, gels, ointments, salves, pastes, powders, solids, and any combination thereof.

9. The sheet assembly of claim 1, wherein the first layer is selected from the group consisting of woven fabrics, extruded sheets, polymeric films, leathers, cellulose products, and any combination thereof.

10. The sheet assembly of claim 1, wherein the medicament in the reservoir is entrained in a substance.

11. The sheet assembly of claim 1, wherein the medicament is contained in a capsule disposed within the inner reservoir.

12. The sheet assembly of claim 1, wherein the first layer is disposed to define a plurality of inner reservoirs attached to form a planar sheet, the medicament being disposed in each inner reservoir.

13. A garment, comprising the sheet assembly of claim 1.

14. The garment of claim 1, further comprising:
a pump mechanism connected to pressurize the inner reservoir.

15. A method of forming a textile assembly, comprising:
disposing an insoluble layer so as to define an inner reservoir; and
disposing a medicament in the inner reservoir.

16. A method for providing a garment, comprising:
disposing an insoluble layer so as to define an inner reservoir;
disposing a medicament in the inner reservoir; and
forming a garment from the insoluble layer containing the medicament.
